(19) United States
(12) Patent Application Publication
(10) Pub. No.: US 2024/0352528 A1
(43) Pub. Date: Oct. 24, 2024

(54) microRNA BIOMARKER FOR EARLY DIAGNOSIS OF MILD COGNITIVE IMPAIRMENT OR ALZHEIMER TYPE DEMENTIA

(71) Applicant: ALZ-DEMENTIA KOREA CO., Gyeongsangnam-do (KR)

(72) Inventors: MYEONG OK KIM, Gyeongsangnam-do (KR); TAE JU PARK, Gyeongsangnam-do (KR); HYUN YOUNG PARK, Gyeongsangnam-do (KR)

(21) Appl. No.: 18/682,176

(22) PCT Filed: Aug. 11, 2022
(86) PCT No.: PCT/KR2022/012056

§ 371 Date: Feb. 8, 2024

(30) Foreign Application Priority Data
Aug. 13, 2021 (KR) 10-2021-0107017
Sep. 3, 2021 (KR) 10-2021-0117316
Nov. 3, 2021 (KR) 10-2021-0149785

(51) Int. Cl. C12Q 1/6883

(52) U.S. Cl. CPC C12Q 1/6883; C12Q 2600/178

(57) ABSTRACT

A microRNA biomarker is for early diagnosis of mild cognitive impairment or Alzheimer type dementia. The microRNA biomarker includes miRNAs of SEQ ID NOs: 1 to 5 obtained by molecule-based analysis are expressed in the blood plasma of patients with mild cognitive impairment or Alzheimer type dementia. The miRNAs have an effect of reducing the expression of proteins that are related to mild cognitive impairment or Alzheimer type dementia. As such, the early diagnosis of mild cognitive impairment or Alzheimer type dementia is enabled before symptoms appear by utilizing a novel miRNA present in relatively non-invasive blood plasma as a diagnostic indicator.

### CROSS REFERENCE TO RELATED APPLICATIONS AND CLAIM OF PRIORITY

This application claims benefit under 35 U.S.C. 119, 120, 121, or 365(c), and is a National Stage entry from International Application No PCT/KR2022/012056 filed on Aug. 11, 2022, which claims priority to the benefit of Korean Patent Application Nos. 10-2021-0107017 filed on Aug. 13, 2021, 10-2021-0117316 filed on Sep. 3, 2021 and 10-2021-0149785 filed on Nov. 3, 2021 in the Korean Intellectual Property Office the entire contents of which are incorporated herein by reference.

### BACKGROUND

### 1. Technical Field

The present invention relates to a novel microRNA biomarker for early diagnosis of mild cognitive impairment or Alzheimer type dementia.

### 2. Background Art

Based on cognitive impairment and the amyloid hypothesis, diagnosis of Alzheimer type dementia is currently made by using neuropsychological tests and diagnostic imaging. However, due to the numerous limitations, efforts are actively made in recent years to develop diagnostic indicators for Alzheimer type dementia from blood, which is a relatively non-invasive sample.

microRNA (miRNA) is a small RNA molecule consisting of about 20 nucleotides in a single strand. As a short RNA molecule, it binds to the 3′-UTR (3′-untranslated region) of specific target mRNA, leading to the inhibition of mRNA translation and thereby regulating protein expression. Although approximately 1,000 miRNAs have been identified in humans until now, the functions of most miRNAs remain undiscovered.

Currently, dementia analysis using blood primarily relies on proteins, but it has significant patient-specific variations in contrast to its structural stability. Additionally, it has the drawback of not fully reflecting the pathology of Alzheimer type dementia in blood. For a more sensitive diagnostic indicator, smaller targets are needed. In the case of Alzheimer type dementia, in particular, abnormalities in protein aggregation occur not only due to genetic factors but also due to various environmental stimuli. Therefore, analyzing changes in gene expression regulation before protein translation, rather than focusing solely on proteins as a product, is considered crucial. When miRNA is employed as a diagnostic indicator, it plays a role in, as a single-stranded RNA consisting of 21 to 25 nucleotides that exists in all biological fluids including blood, urine, and saliva, directly regulating gene expression by inhibiting the translation of mRNA. Furthermore, being resistant to degradation by RNase, miRNA remains stable in blood, and defects in the metabolism or function of miRNA are known to be closely related with the onset of dementia, showing high specificity and sensitivity depending on the type of disease. Thus, miRNA can be utilized as the most optimal diagnostic indicator.

Diagnosis of Alzheimer's disease is crucially dependent on accurate history-taking through reports from caregivers who know the patient best. Physicians inquire about changes in cognitive function, including memory, compared to the past, and examine when and how those changes manifested. Diagnosis involves a combination of physical examination, neurological examination, mental status examination, assessment of daily living functions, biochemical tests such as blood tests, neuroimaging tests, and neuropsychological tests. Despite these approaches, diagnosing Alzheimer's disease still presents several limitations, making early diagnosis challenging. Currently, Alzheimer type dementia is primarily diagnosed based on cognitive impairment and the amyloid hypothesis, employing neuropsychological tests and diagnostic imaging. In recent years, there is a growing effort to develop indicators for diagnosing Alzheimer type dementia from blood as non-invasive sample.

Meanwhile, miRNA is a small RNA with a single strand of about 20 nucleotides. As a short RNA molecule, it binds to the 3′-UTR of specific target mRNA, leading to the inhibition of mRNA translation and thereby regulating protein expression. Although approximately 1,000 miRNAs have been identified in humans until now, the functions of most miRNAs remain undiscovered.

Currently, dementia analysis using blood primarily relies on proteins, but it has significant patient-specific variations in contrast to its structural stability. Additionally, it has the drawback of not fully reflecting the pathology of Alzheimer type dementia in blood. For a more sensitive diagnostic indicator, smaller targets are needed. In the case of Alzheimer type dementia, in particular, abnormalities in protein aggregation occur not only due to genetic factors but also due to various environmental stimuli. Therefore, analyzing changes in gene expression regulation before protein translation, rather than focusing solely on proteins as a product, is considered crucial.

For instance, when miRNA is employed as a diagnostic indicator, it plays a role in, as a single-stranded RNA consisting of 21 to 25 nucleotides that exists in all biological fluids including blood, urine, and saliva, directly controlling gene expression by inhibiting the translation of mRNA. Furthermore, being resistant to degradation by RNase, miRNA remains stable in blood, and defects in the metabolism or function of miRNA are known to be closely related with the onset of dementia, showing high specificity and sensitivity depending on the type of disease. Thus, miRNA can be utilized as the most optimal diagnostic indicator.

Meanwhile, technologies related to the diagnosis of cognitive impairment include Korean Patent Registration No. 1992539, which discloses a composition and a method for diagnosing cognitive impairment disorders based on miRNA. Additionally, Korean Patent Registration No. 1718940 discloses an epigenetic early diagnostic composition for Alzheimer type dementia or mild cognitive impairment. However, there is no disclosure yet regarding the novel microRNA biomarker for early diagnosis of mild cognitive impairment or Alzheimer type dementia as presented in the present invention.

### SUMMARY

The present invention is devised under the circumstances that are described in the above and it provides a novel microRNA biomarker for early diagnosis of mild cognitive impairment or Alzheimer type dementia. It was found that the novel miRNAs represented by SEQ ID NO: 1 to SEQ ID NO: 5 according to the present invention are expressed in the blood plasma of patients with mild cognitive impairment and also in the blood plasma of patients with mild cognitive impairment or Alzheimer type dementia, enabling the early diagnosis of mild cognitive impairment and Alzheimer type dementia, and those novel miRNAs can reduce the expression of Alzheimer type dementia-related proteins, thus the present invention is completed accordingly.

To achieve the object described in the above, the present invention provides a biomarker composition for early diagnosis of mild cognitive impairment or Alzheimer type dementia comprising any one of miRNAs selected from the group consisting of the nucleotide sequences of SEQ ID NOs: 1 to 5 as an effective component.

The present invention further provides a biosensor composition for early diagnosis of mild cognitive impairment or Alzheimer type dementia comprising an agent capable of detecting any one of miRNAs selected from the group consisting of the nucleotide sequences of SEQ ID NOs: 1 to 5 as an effective component.

The present invention further provides a kit for early diagnosis of mild cognitive impairment or Alzheimer type dementia including the aforementioned biosensor composition for early diagnosis of mild cognitive impairment or Alzheimer type dementia.

The present invention further provides a method of providing information for early diagnosis of mild cognitive impairment or Alzheimer type dementia including:
(1) collecting blood from an individual,
(2) separating blood plasma from the blood of the above step (1); and
(3) examining whether any one of miRNAs selected from the group consisting of the nucleotide sequences of SEQ ID NOs: 1 to 5 in the blood plasma separated in the above step (2) is expressed or not.

The present invention still further provides a method for early diagnosis of mild cognitive impairment or Alzheimer type dementia including:
(1) collecting blood from an individual,
(2) separating blood plasma from the blood of the above step (1); and
(3) examining whether any one of miRNAs selected from the group consisting of the nucleotide sequences of SEQ ID NOs: 1 to 5 in the blood plasma separated in the above step (2) is expressed or not.

The present invention relates to a novel microRNA biomarker for early diagnosis of mild cognitive impairment or Alzheimer type dementia. Specifically, miRNAs of SEQ ID NOs: 1 to 5 according to the present invention are expressed in the blood plasma of patients with mild cognitive impairment/Alzheimer type dementia, and they have an effect of reducing the expression of proteins that are related with Alzheimer type dementia.

As such, the present invention has the advantage of enabling the early diagnosis of mild cognitive impairment or Alzheimer type dementia before symptoms appear by utilizing a novel miRNA present in relatively non-invasive blood plasma as a diagnostic indicator.

### BRIEF DESCRIPTION OF THE DRAWINGS

FIG. 1 represents the RNA constitution of each patient sample, showing the proportion of RNA types classified from the reads.

FIG. 2 illustrates the stem-loop structure and sequence information of the novel miRNA determined in the present invention (SEQ ID NO: 1).

FIG. 3 illustrates the stem-loop structure and sequence information of the novel miRNA determined in the present invention (SEQ ID NO: 2).

FIG. 4 illustrates the stem-loop structure and sequence information of the novel miRNA determined in the present invention (SEQ ID NO: 3).

FIG. 5 illustrates the stem-loop structure and sequence information of the novel miRNA determined in the present invention (SEQ ID NO: 4).

FIG. 6 illustrates the stem-loop structure and sequence information of the novel miRNA determined in the present invention (SEQ ID NO: 5).

FIG. 7 depicts the mimic miRNA1 (SEQ ID NO: 1) and miRNA inhibitor synthetic sequence (SEQ ID NO: 6) for determining the function of the novel miRNAs of the present invention.

FIGS. 8A and 8B depict the mimic and inhibitor synthetic sequences of miRNA2 (SEQ ID NO: 2) (FIG. 8A) and miRNA3 (SEQ ID NO: 3) (FIG. 8B) for determining the function of the novel miRNAs of the present invention.

FIGS. 9A and 9B depict the mimic and inhibitor synthetic sequences of miRNA4 (SEQ ID NO: 4) (FIG. 9A) and miRNA5 (SEQ ID NO: 5) (FIG. 9B) for determining the function of the novel miRNAs of the present invention.

FIG. 10 presents the Western blot results for determining the changes in the expression level of SYNJ1, SNAP91, and HOOK3 as a target gene protein of the novel miRNA1 of the present invention. *, **, and *** indicate statistically significant differences in the expression level of target genes between the untreated control group and the groups treated with mimic miRNA of the present invention or miRNA inhibitor, in which * represents p<0.05, ** represents p<0.01, and *** represents p<0.001.

FIG. 11 presents the result of determining the changes in the expression level of SYNJ1, SNAP91, and HOOK3 as a target gene protein of the novel miRNA1 of the present invention, in which the changes were determined by immunocytochemistry.

FIG. 12 presents the Western blot results for determining the changes in the expression level of BDNF, NRG3, and NEFL as a target gene protein of the novel miRNA2 of the present invention. *, **, and *** indicate statistically significant differences in the expression level of target genes between the untreated control group and the groups treated with mimic miRNA of the present invention or miRNA inhibitor, in which * represents p<0.05, ** represents p<0.01, and *** represents p<0.001.

FIG. 13 presents the Western blot results for determining the change in the expression level of HOOK3 as a target gene protein of the novel miRNA3 of the present invention. * and *** indicate statistically significant differences in the expression level of target gene between the untreated control group and the groups treated with mimic miRNA of the present invention or miRNA inhibitor, in which * represents p<0.05 and *** represents p<0.001.

FIG. 14 presents the result of determining the change in the expression level of HOOK3 as a target gene protein of the novel miRNA3 of the present invention, in which the change was determined by immunocytochemistry.

FIG. 15 presents the Western blot results for determining the change in the expression level of MAP1A (microtubule associated protein 1A) that is encoded by the target gene of the novel miRNA4 of the present invention. ** and *** indicate statistically significant differences in the expression level of target genes between the untreated control group and the groups treated with mimic miRNA of the present invention or miRNA inhibitor, in which ** represents p<0.01 and *** represents p<0.001.

FIG. 16 presents the Western blot results for determining the change in the expression level of SHANK1 (SH3 and multiple ankyrin repeat domains protein 1) that is encoded by the target gene of the novel miRNA5 of the present invention. ** and ** indicate statistically significant differences in the expression level of target genes between the untreated control group and the groups treated with mimic miRNA of the present invention or miRNA inhibitor, in which ** represents p<0.01 and *** represents p<0.001.

FIG. 17 presents the result of determining the changes in the expression level of MAP1A and SHANK1, encoded by the novel miRNA4 and miRNA5 of the present invention, respectively, in the blood plasma of the mild cognitive impairment group (MCI) and Alzheimer dementia group (AD). * and *** indicate statistically significant differences in the expression level of target proteins between the mild cognitive impairment group (MCI) and Alzheimer dementia group (AD), in which * represents p<0.05 and *** represents p<0.001.

### DETAILED DESCRIPTION

The present invention relates to a biomarker composition for early diagnosis of mild cognitive impairment or Alzheimer type dementia comprising any one of miRNAs selected from the group consisting of the nucleotide sequences of SEQ ID NOs: 1 to 5 as an effective component

In the present invention, microRNA (miRNA) refers to 21 to 23 nucleotide-long non-coding RNAs that regulate gene expression post-transcriptionally by promoting the degradation of target RNAs or inhibiting their translation. Included in the miRNA are not only miRNAs represented by specific nucleotide sequences but also their precursors (pre-miRNA, pri-miRNA), miRNAs that are biologically equivalent to them (namely, homologs or isologs), variant forms such as those with genetic polymorphism, and derivatives.

The miRNAs are preferably those derived from human blood plasma, but they are not limited thereto.

The miRNA of SEQ ID NO: 1 is characterized in that it can reduce the gene expression level of SYNJ1 (synaptojanin 1), SNAP91 (synaptosome associated protein 91), or HOOK3 (hook microtubule tethering protein 3).

The miRNA of SEQ ID NO: 2 is characterized in that it can reduce the gene expression level of BDNF (Brain-Derived Neurotrophic Factor), NRG3 (Neuregulin3), and NEFL (Neurofilament Light Chain) and miRNA of SEQ ID NO: 3 is characterized in that it can reduce the gene expression level of HOOK3 (Protein Hook homolog 3).

The miRNA of SEQ ID NO: 4 is characterized in that it can reduce the gene expression level of MAP1A (microtubule associated protein 1A) and miRNA of SEQ ID NO: 5 is characterized in that it can reduce the gene expression level of SHANK1 (SH3 and multiple ankyrin repeat domains protein 1).

The miRNAs of SEQ ID NO: 4 and SEQ ID NO: 5 have the characteristic of enabling selective diagnosis of mild cognitive impairment and Alzheimer type dementia. As described herein, the expression “selective diagnosis” refers not only to the early diagnosis of mild cognitive impairment and Alzheimer type dementia but also to the ability to distinguish between mild cognitive impairment and Alzheimer type dementia.

The expression “diagnosis” described herein encompasses determining the susceptibility of an individual (i.e., test subject) to a specific disease or disorder, determining whether an individual currently has a specific disease or disorder or not, making a prognosis for an individual afflicted with a specific disease or disorder, and employing theranostics (for example, monitoring the condition of an individual to provide information on therapeutic efficacy).

The present invention further relates to a biosensor composition for early diagnosis of mild cognitive impairment or Alzheimer type dementia comprising an agent capable of detecting any one of miRNAs selected from the group consisting of the nucleotide sequences of SEQ ID NOs: 1 to 5 as an effective component.

The aforementioned agent may contain a nano-fluorescent complex having biotin-streptavidin serving as a linker and a fluorophore of various wavelengths and a quencher bound to the complex.

The aforementioned agent may be an antisense oligonucleotide, a primer, or a probe complementary to the miRNAs.

Mild cognitive impairment refers to an intermediate stage between normal age-related cognitive decline and dementia. Namely, it signifies a state in which cognitive abilities are diminished compared to the same age group, yet individuals can still perform activities of daily living, distinguishing it from dementia. Mild cognitive impairment is considered an intermediate stage between normalcy and dementia, and known to be a high-risk group for dementia.

Therefore, in the present invention, as the test group for early diagnosis of mild cognitive impairment or Alzheimer type dementia, the ‘mild cognitive impairment or Alzheimer type dementia patient group’ was employed.

The present invention further relates to a kit for early diagnosis of mild cognitive impairment or Alzheimer type dementia including the aforementioned biosensor composition for early diagnosis of mild cognitive impairment or Alzheimer type dementia.

The aforementioned kit is preferably a kit used for RT-PCR (real-time polymerase chain reaction), real-time PCR, isothermal PCR, Northern blotting, RNA protection assay, or microarray chip, but it is not limited thereto.

The kit of the present invention may further include a nucleic acid that can specifically bind to any one of miRNAs selected from the group consisting of the nucleotide sequences of SEQ ID NOs: 1 to 5. The expression “nucleic acid” encompasses RNA, DNA, and RNA/DNA (chimera), and the DNA may include cDNA, genomic DNA, and synthetic DNA.

Additionally, the aforementioned RNA may encompass total RNA, mRNA, rRNA, miRNA, siRNA, snoRNA, snRNA, non-coding RNA, and synthetic RNA. The kit of the present invention may also include, in addition to the aforementioned nucleic acids, nucleic acids that enable the early diagnosis of Alzheimer type dementia or include nucleic acids that can be discovered in the future. The kit may also include antibodies known for measuring markers related to the early diagnosis of Alzheimer type dementia. The nucleic acids included in the kit can be packaged in different containers, either individually or in any combination of them.

The kit of the present invention may include fluorescent materials for labeling, enzymes and culture media for nucleic acid amplification, and a user manual. The kit of the present invention serves as a device for early diagnosis of Alzheimer type dementia, in which the nucleic acids are linked or attached to a solid support, for example. Examples of materials for the solid support include plastic, paper, glass, and silicone, and plastic is desirable for its ease of processing. The solid support can have any shape, such as square, circular, rectangular, or film-shaped.

The kit of the present invention can be included in a medical device for HTS (high-throughput-screening)-based multiplexed POCT (point-of-care-testing). A cartridge within the medical device may consist of an incubator, a stirrer, a disposable tip, and a sample injection container, and, as an all-in-one medical device equipped with a micro whole blood separator, a fluorescence detector, and a liquid handler, an algorithm and an automated detection system for automatically generating ‘dementia risk index’ upon injection of human blood may be also included.

The present invention further relates to a method of providing information for early diagnosis of mild cognitive impairment or Alzheimer type dementia including:
(1) collecting blood from an individual,
(2) separating blood plasma from the blood of the above step (1); and
(3) examining whether any one of miRNAs selected from the group consisting of the nucleotide sequences of SEQ ID NOs: 1 to 5 in the blood plasma separated in the above step (2) is expressed or not.

The present invention still further relates to a method for early diagnosis of mild cognitive impairment or Alzheimer type dementia including:
(1) collecting blood from an individual,
(2) separating blood plasma from the blood of the above step (1); and
(3) examining whether any one of miRNAs selected from the group consisting of the nucleotide sequences of SEQ ID NOs: 1 to 5 in the blood plasma separated in the above step (2) is expressed or not.

Hereinbelow, the present invention is explained in greater detail in view of Examples. However, the following Examples are given only for more specific explanation of the present invention and it is evident to a person who has common knowledge in the pertinent art that the scope of the present invention is not limited by them.

### EXAMPLES

### 1. Preparation of Test Sample From Patient Having Alzheimer Type Dementia

From a group of patients who had been previously classified based on the severity of Alzheimer type dementia through selective diagnostic tools (K-MMSE and SNSB) score and imaging diagnoses (PET, fMRI, and CT), blood was collected from the patients, i.e., 10 people for each patient group, who have been found to have mild cognitive impairment (MCI) or Alzheimer type dementia (AD), and blood plasma was separated from the blood. The separated blood plasma was stored under freezing condition, i.e. at −70° C., until use. Meanwhile, as a control group, blood was collected from 10 healthy individuals, and their blood plasma was also separated and stored under freezing condition, i.e. at −70° C., until use, similar to the patient group.

### 2. Extraction of Total miRNA

Total RNA present in the obtained blood plasma of each of the patient group and control group was extracted using the QIAGEN miRNeasy Serum/Plasma Separation Kit, which is mainly used for purifying miRNA and other small RNAs from small amounts of serum and blood plasma.

Subsequently, by using the Bioanalyzer RNA Small RNA chip, the amount of obtained RNA was determined to ensure that it is sufficient for library construction for NGS analysis.

### 3. Analysis of NGS (Next Generation Sequencing)

Small RNA-seq library was generated according to the Illumina Platform by using the SMARTer smRNA-Seq Kit, which allows the preparation of high-quality miRNA library even from small amount of RNA. Following the manual of SMARTer smRNA-Seq Kit, the prepared sample was sequenced on the NovaSeq 6000 sequencing platform.

### 4. Selection of miRNA Data Through Analysis of Unprocessed Raw Data

The unprocessed raw data obtained from NGS analysis were processed by trimming in the order of Adapter, Quality, and Length to remove contaminated or low-abundant data present in the blood plasma. After this trimming process, miRNA Deep2* sequencing was performed with the resultant clean data in which unnecessary information has been eliminated.

### 5. miRNA Deep2* Sequencing

miRNA Deep2* is a software that can identify novel miRNA from purified but unprocessed raw data. According to the RNAfold algorithm, it predicts novel miRNA by aligning the mature, star, and loop sequences with the short-cut miRBase (Pre-miRNA) library. The RNAfold algorithm can derive the secondary structure with the minimum free energy for RNA sequence by using the most similar thermodynamic model. The RNA fold generated graphic includes read counts for each part of the hairpin, scores for minimum free energy, scores for randfold, and scores for conserved seed sequence.

The miRDeep2* system can detect both previously known miRNAs and novel miRNAs based on mapping using RNA database.

### 6. Determination of Novel Alzheimer Type Dementia-Related miRNA Reflected in Blood

To determine the functional relationship of the novel miRNA with Alzheimer type dementia, screening was carried out by using the miRDB software, which performs scoring of target genes (miRNA) based on the sequence of miRNA. Determination was made based on the analysis of expression level of the genes associated with Alzheimer type dementia among the target genes with high genetic scores.

Specifically, SH-SY5Y cells, commonly used as a model for neural function and differentiation, were utilized. After the transfection of SH-SY5Y cells with miRNA mimic which exhibits overexpression effects of the selected novel miRNA, miRNA inhibitor which inhibits the expression of novel miRNA, or a negative control miRNA, protein expression of the target gene was examined through Western blot analysis.

### Example 1. Preparation of Test Sample and Extraction of Total RNA

Using the QIAGEN miRNeasy Serum/Plasma Kit which allows the purification of miRNA and other small RNA molecules from small amounts of serum and blood plasma, total RNA present in each blood plasma, which has been collected from both the patient group and the control group, was extracted. Subsequently, RNA quantification was carried out by using the Bioanalyzer RNA Small RNA chip to ensure that a sufficient amount of RNA was obtained for library preparation for NGS analysis. As a result, it was found that the RNA was obtained in a sufficient amount for NGS analysis

### Example 2. Next Generation Sequencing (NGS)

### (1) Library Construction and Obtainment of Unprocessed Raw Data

For generating the small RNA-seq library for sequencing on the Illumina platform for NGS, SMARTer smRNA-Seq Kit, which allows the construction of high-quality miRNA libraries even from small amount of RNA, was employed. Library for sequencing total RNA obtained from the blood plasma of both the patient group and control group was constructed. By carrying out sequencing using the constructed library and NovaSeq 6000 sequencing platform, unprocessed raw data were obtained.

(2) Selection of miRNA Data From Unprocessed Raw Data

The unprocessed raw data obtained from the NGS analysis were subjected to trimming in the order of Adapter, Quality, and Length to remove contaminated or low-abundant data.

Specifically, the presence or absence of Adapter (nonAdapter Read Count), appropriate length (Short Read Count), and degree of low quality (Low Quality Read Count) were determined from the Total Read Count as the unprocessed raw data obtained through sequencing. The refined results obtained from a group of 10 patients are presented in Table 1. Using this primary processed data, which had unnecessary information removed through the trimming and selection process, miRNA Deep2* sequencing was then carried out.

TABLE 1
Highly accurate data having unnecessary information
removed through trimming and selection process

| | Total | Reain | | Filtered | |
|---|---|---|---|---|---|
| Sample | Read Count | Read Count | | Read Count | |
| 1 | 27,219,398 | 18,390,807 | (67.57%) | 8,828,591 | (32.43%) |
| 2 | 26,218,999 | 11,956,632 | (45.6%) | 14,262,367 | (54.4%) |
| 3 | 26,571,228 | 16,969,474 | (63.86%) | 9,601,754 | (36.14%) |
| 4 | 23,724,381 | 15,139,175 | (63.81%) | 8,585,206 | (36.19%) |
| 5 | 22,907,556 | 14,289,335 | (62.38%) | 8,618,221 | (37.62%) |
| 6 | 16,222,537 | 6,377,318 | (39.31%) | 9,845,219 | (60.69%) |
| 7 | 20,776,579 | 9,679,288 | (46.59%) | 11,097,291 | (53.41%) |
| 8 | 17,732,489 | 9,912,646 | (55.9%) | 7,819,843 | (44.1%) |
| 9 | 7,664,012 | 3,538,318 | (46.17%) | 4,125,694 | (53.83%) |
| 10 | 28,799,084 | 19,621,616 | (68.13%) | 9,177,468 | (31.87%) |

(3) miRNA Deep2* Sequencing

miRNA Deep2* is a software that can identify new miRNA from the above primary processed data using the RNAfold algorithm. Specifically, novel miRNA was predicted by aligning in parallel the mature, star, and loop sequences with the short-cut miRBase (Pre-miRNA) library. The RNAfold algorithm uses the most similar thermodynamic model to predict the secondary structure of RNA sequence with the minimum free energy.

RNA fold generated graphics include read counts for each part of the hairpin, scores for minimum free energy, scores for randfold, and scores for conserved seed sequences. Based on mapping utilizing RNA database, the miRDeep2* system allows the detection of both previously-known miRNA and novel miRNA.

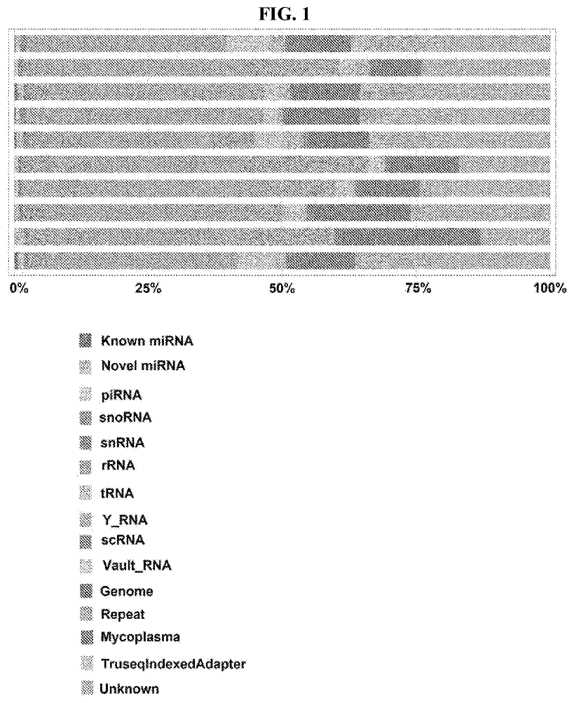

The final processed reads for the above 10 test samples were classified into piRNA, tRNA, snRNA, snoRNA, previously known miRNA, and novel miRNA by using miRBase v22.1 and the non-coding RNA database (RNAcentral release 14.0) (FIG. 1).

The genome mapping was carried out by using RSEM with Bowtie and STAR. Bowtie was subsequently used with genome sequences for miRDeep2 analysis. Known/novel miRNAs predicted by miRDeep2 and other small RNAs matched with RNAcentral were aligned using Bowtie (target small RNAs, <50 nt) and Bowtie2 (target smRNA, ≥50 nt).
RSEM (RNA-Seq by Expectation-Maximization): Tool for quantifying the RNA-Seq transcript
Bowtie: Software commonly used for sequence alignment and sequence analysis
STAR (Spliced Transcripts Alignment to a Reference): Fast RNA-seq read mapper

To predict novel miRNAs, uniquely clustered reads were separately aligned with the reference genome and precursor miRNAs. The mature star and loop sequences of the miRNA can be predicted according to the RNAfold algorithm using miRNA Deep2* software. The RNAfold function enables prediction of the minimum free energy secondary structure of RNA sequence by using the nearest-neighbor thermodynamic model. The graphical output generated by RNAfold includes the actual in silico-folded hairpin, with read counts for each part of the hairpin, minimum free energy scores, randfold scores, and scores for conserved seed sequences.

With scores ranging from −10 to 10, miRDeep2* allowed obtainment of novel miRNAs. The obtained novel miRNAs are expressed in both the mild cognitive impairment group and the Alzheimer's disease group. Among them, two types of the miRNAs with miRDeep2* scores equal to or greater than 1, a “yes” designation in RNAfold, and a mature read count of 10 or more were selected.

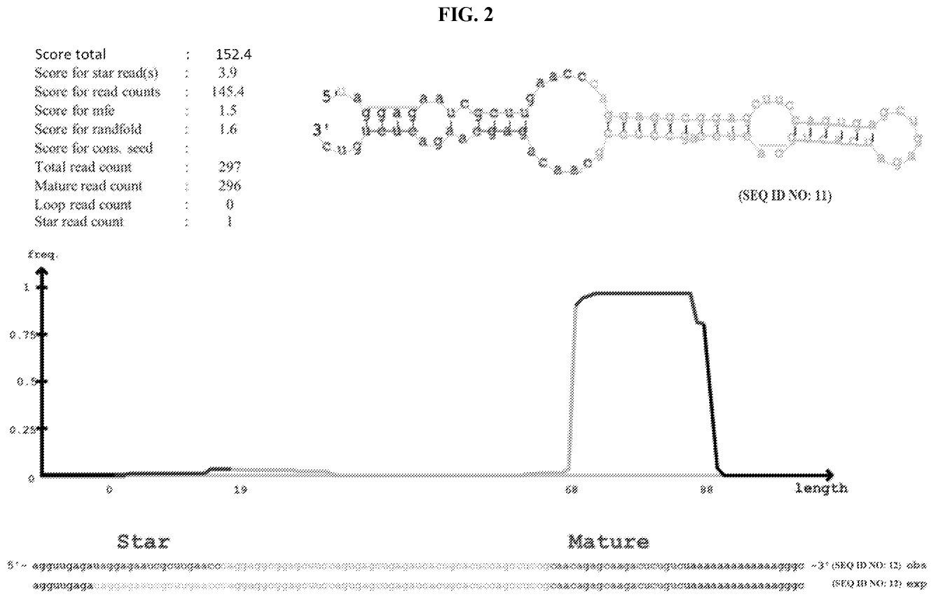

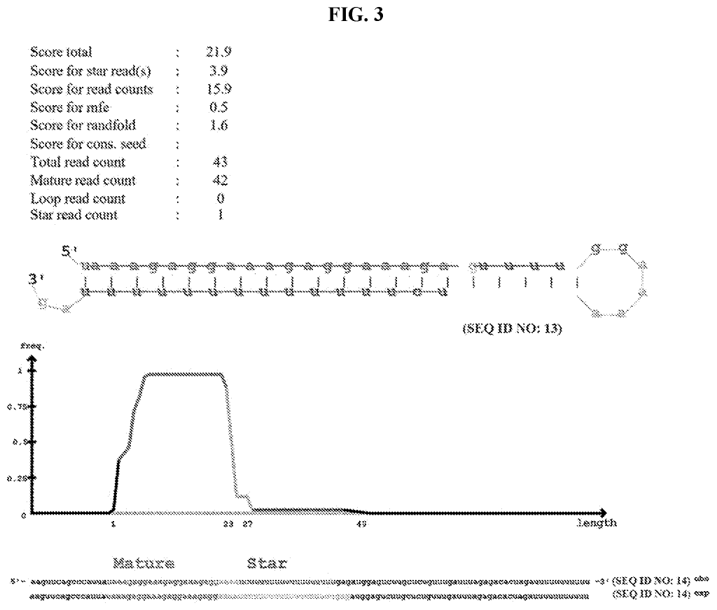

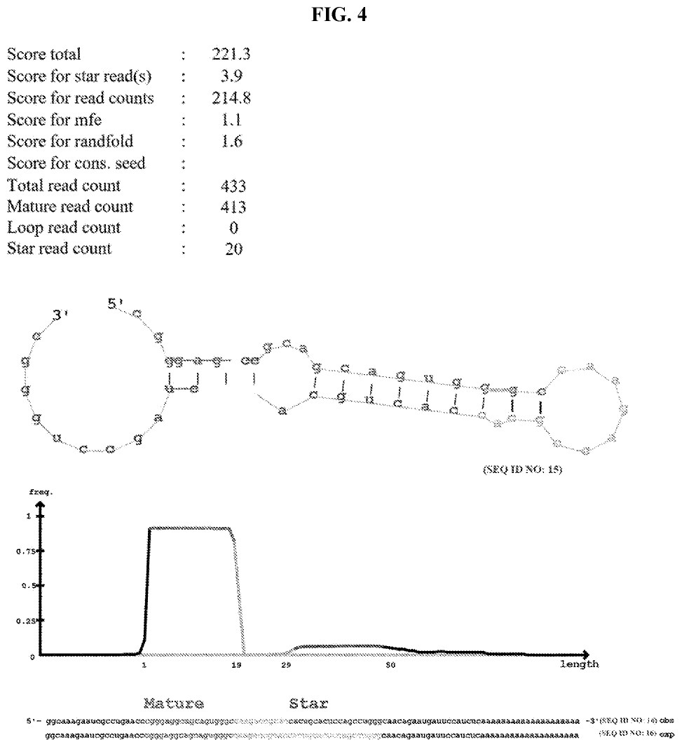

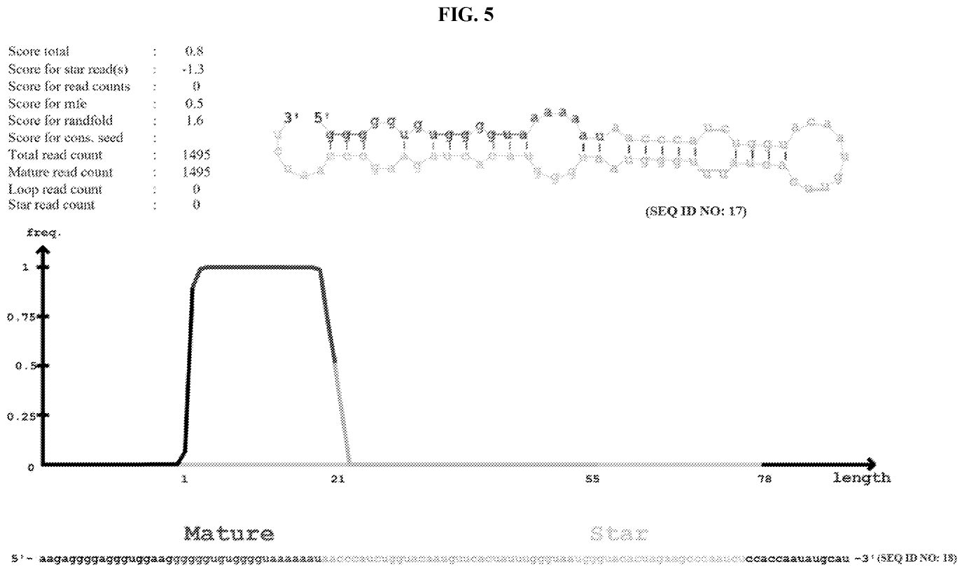

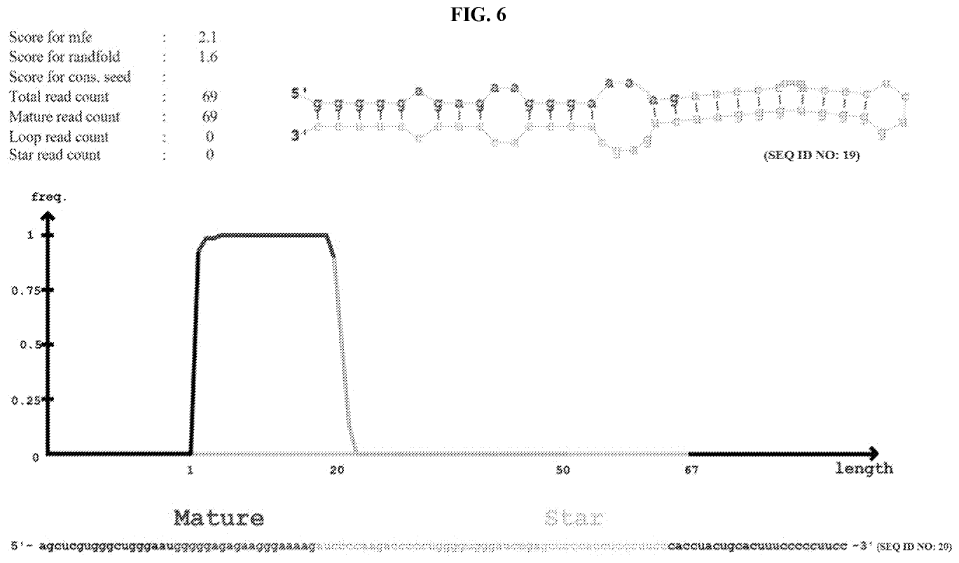

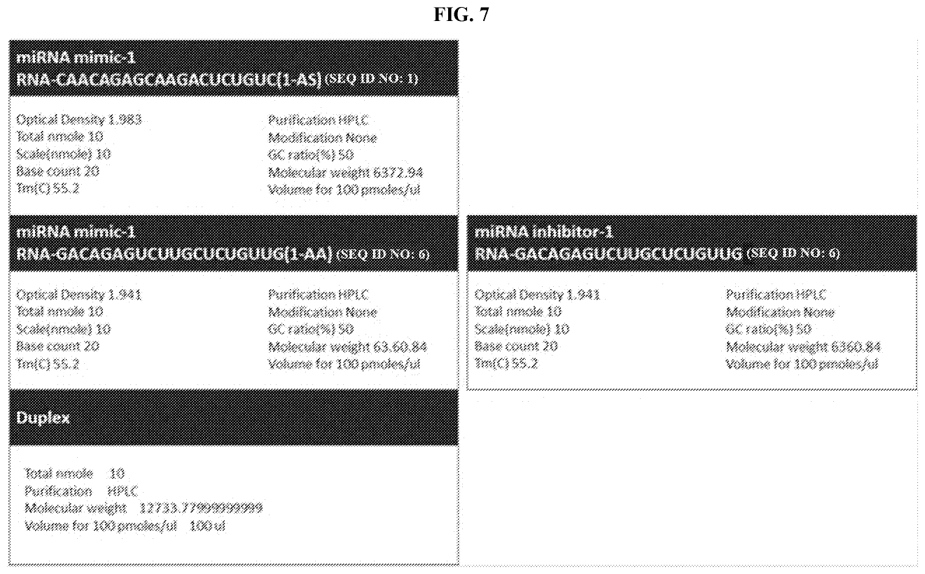

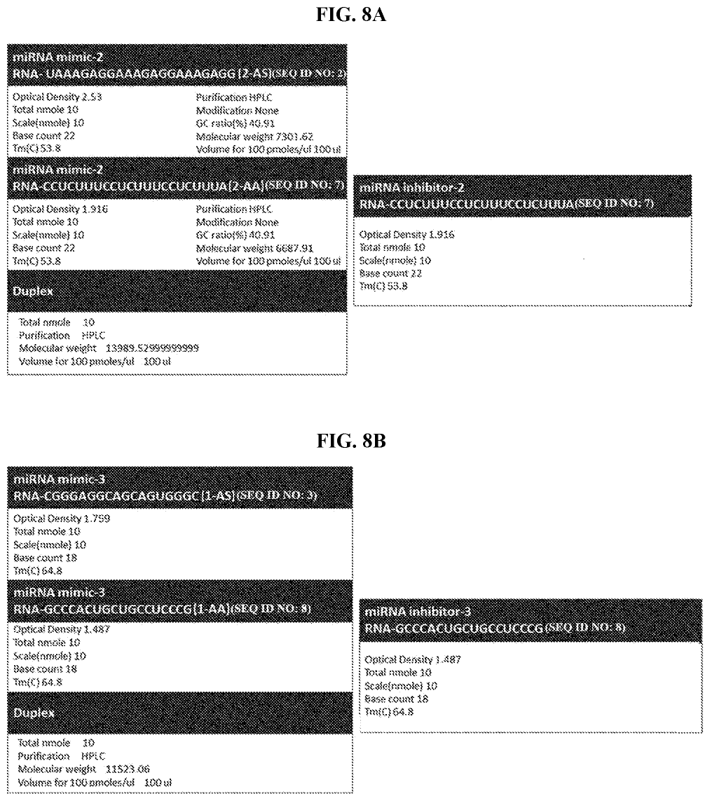

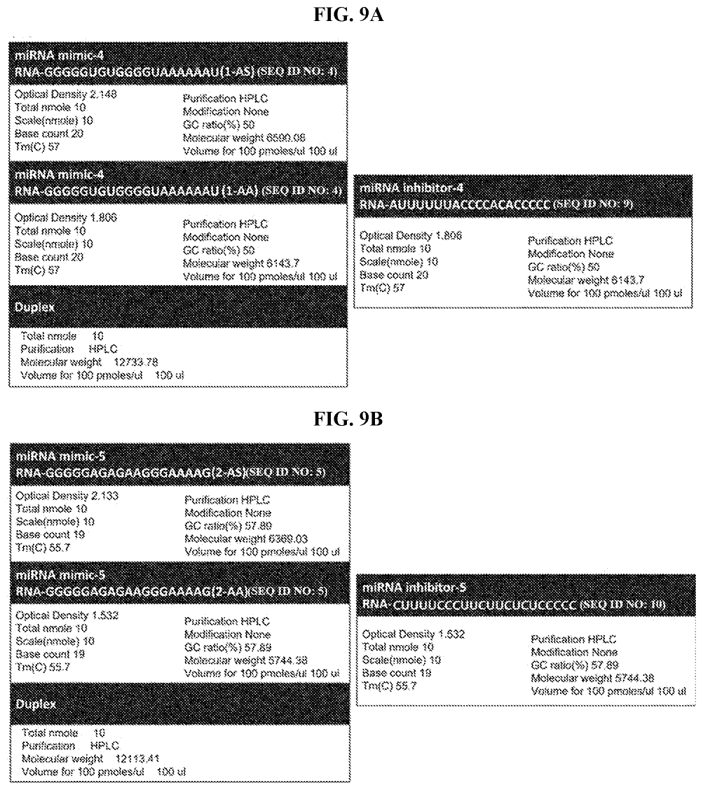

Stem loop structure of the selected novel miRNAs, namely miRNA2, miRNA3, miRNA4, and miRNA5, are depicted in FIGS. 2 to 6. Furthermore, their sequence information is provided in FIGS. 7 to 9B.

### Example 3. Examination of Novel miRNA's Property of Regulating Expression Level of Alzheimer Type Dementia-Related Genes

To determine the relationship of the novel miRNA with Alzheimer type dementia, miRDB software to screen by scores for target genes based on the sequence of miRNA was utilized. Among the target genes showing high gene scores, those associated with Alzheimer type dementia were selected.

SH-SY5Y cells, commonly used as a model for neural function and differentiation, were employed. After the transfection of the SH-SY5Y cells with miRNA mimic which exhibits overexpression effects of the selected novel miRNA, miRNA inhibitor which inhibits the expression of novel miRNA, or a negative control miRNA, protein expression of the target gene was examined through Western blot analysis.

The miRNA mimics are miRNAs that can function similarly to the novel miRNAs in living human body, and they were prepared as a double strand (specifically, the sequence of miRNA 1 mimic is 5′-CAACAGAGCAAGACUCUGUC-3′ (1-AS) (SEQ ID NO: 1) and the miRNA inhibitor for miRNA 1 mimic of SEQ ID NO: 1 is 5′-GACAGAGUCUUGCUCUGUUG-3′ (1-AA) (SEQ ID NO: 6); the sequence of miRNA2 mimic is 5′-UAAAGAGGAAAGAGGAAAGAGG-3′ (SEQ ID NO: 2) and the miRNA inhibitor for miRNA2 mimic of SEQ ID NO: 2 is 5′-CCUCUUUCCUCUUUCCUCUUUA-3′ (SEQ ID NO: 7); the sequence of miRNA3 mimic is 5′-CGGGAGGCAGCAGUGGGC-3′ (SEQ ID NO: 3) and the miRNA inhibitor for miRNA3 mimic of SEQ ID NO: 3 is 5′-GCCCACUGCUGCCUCCCG-3′ (SEQ ID NO: 8); the sequence of miRNA 4 mimic is 5′-GGGGGUGUGGGGUAAAAAAU-3′ (SEQ ID NO: 4) and the miRNA inhibitor for miRNA 4 mimic of SEQ ID NO: 4 is 5′-AUUUUUUACCCCACACCCCC-3′ (SEQ ID NO: 9); and the sequence of miRNA 5 mimic is 5′-GGGGGAGAGAAGGGAAAAG-3′ (SEQ ID NO: 5) and the miRNA inhibitor for miRNA 5 mimic of SEQ ID NO: 5 is 5′-CUUUUCCCUUCUUCUCUCCCCC-3′ (SEQ ID NO: 10)).

The miRNA inhibitor has a sequence that is complementary to the sequence of the obtained miRNA, and, as prepared in a single bond, it is a miRNA capable of inhibiting the corresponding miRNA. On the other hand, the negative control miRNA is miRNA consisting of miRNAs not regulating any genes and serves as a reference for determination. The prepared mimics of novel miRNA1 to miRNA5; and miRNA inhibitors for each of them are described in FIGS. 7 to 9B.

[Gene Screening Using miRDB Software]

Among the gene regulated by the novel miRNAs, in order to select a target that is highly relevant to Alzheimer type dementia, genes were screened for each score by using target-predicting miRDB software based on the novel miRNA sequence 5′-CAACAGAGCAAGACUCUCUCUGUC-3′ (SEQ ID NO: 1). After setting the cut-off value of the gene score of the miRNA to 80, three types of genes as miRNA target gene related to neural and synaptic function were selected, i.e., SYNJ1 (synaptojanin 1), SNAP91 (synaptosome associated protein 91), and HOOK3 (hook microtubule tethering protein 3).

Functions of the other candidate target genes are as follows. SYNJ1 plays an important role in synaptic vesicle recirculation. SNAP91 is involved in synaptic vesicle transport and movement and plays a key role in synaptic transmission. HOOK3 is a cytoplasmic linker protein involved in endosome transport and is expressed primarily in neurons. It was possible to find that the novel miRNAs are related to the synaptic proteins in nervous system.

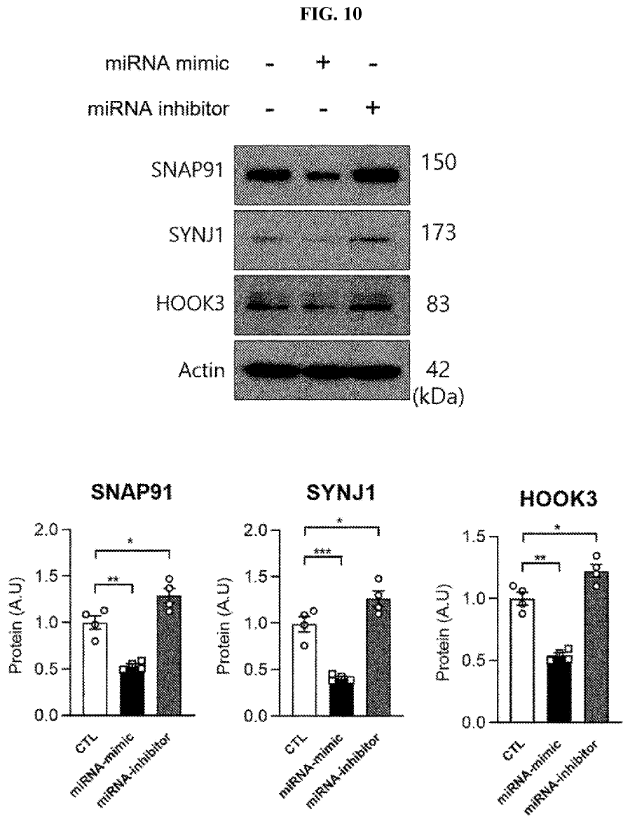

miRNA mimic for the overexpression of novel miRNA and miRNA inhibitor for functional inhibition were separately transfected into human neuroblastoma cells (SH-SY5Y), each at a concentration of 100 nM, for 24 hours. After that, protein separation was carried out, and the expression level was verified using Western blot analysis. The results showed a significant decrease in the expression of candidate target gene proteins, SYNJ1, SNAP91, and HOOK3, as caused by an increase in miRNA binding to 3′ due to the overexpression of the novel miRNA. Conversely, when inhibitor miRNA was applied to remove the miRNA binding at the 3′, the expression of SYNJ1, SNAP91, and HOOK3 proteins increased (FIG. 10).

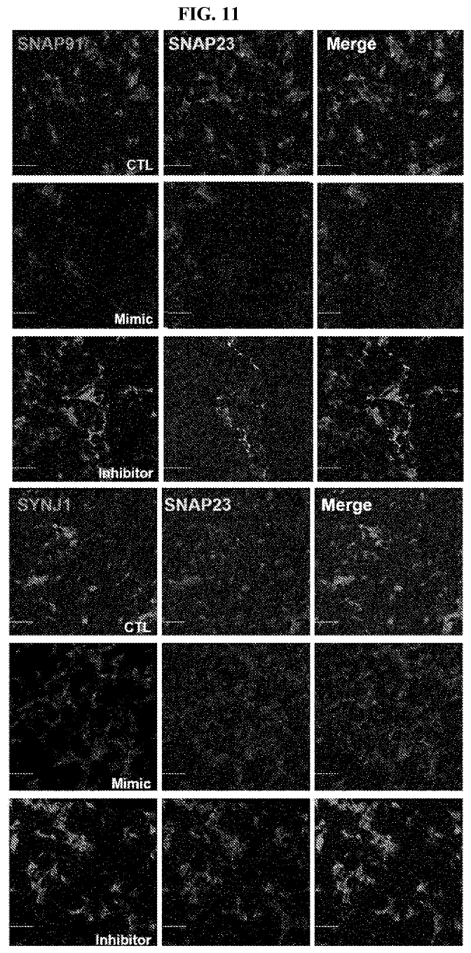

Moreover, the expression of target gene proteins regulated by the mimic miRNAs and inhibitor miRNAs was compared using immunocytochemistry. SNAP23 was identified as the positive control, and both SNAP91 and SYNJ1 exhibited expression patterns that are consistent with Western blot results (FIG. 11).

Meanwhile, among the novel miRNAs, miRNA2 mimic was screened for each score by using target prediction miRDB software, based on the sequence 5′-UAAAGAGGAAGGGAAGGGAAGAGG-3′ (SEQ ID NO: 2), and miRNA3 mimic was also screened for each score based on sequence 5′-CGGGAGGCAGUGGGC-3′ (SEQ ID NO: 3). The miRNA inhibitor for miRNA mimic of SEQ ID NO: 2 is 5′-CCUCUUUCCUCUCUCUUUA-3′ (SEQ ID NO: 7), and the miRNA inhibitor for miRNA mimic of SEQ ID NO: 3 is 5′-GCCCACUGCCUCCCG-3′ (SEQ ID NO: 8). After setting the cut-off value of the gene score of the miRNA to 80, as the miRNA2 target gene related to neural and synaptic function, BDNF (Brain-Derived Neurotrophic Factor), which is essential as neurotrophins for the molecular mechanism of synaptic transmission and synaptic plasticity as well as neuronal survival and differentiation in the central nervous system, NEFL (Neurofilament Light Chain), which is responsible for increasing the structural stability of neurons by constituting neurofilaments in the neuronal cell cytoplasm and, being associated with axon damage, recently been identified as a biomarker of degenerative brain diseases, and NRG3 (Neuregulin3), which regulates the growth and differentiation of epithelial and glial cells and plays an important role in the development, maintenance, and repair of the nervous system, were selected, and, as the target gene of novel miRNA3, the target gene HOOK3 (Protein Hook homolog 3), which is a cytoplasmic linker gene associated with endosomal transport in neurons and related to the production of amyloid beta, the causative protein of Alzheimer type dementia, was selected.

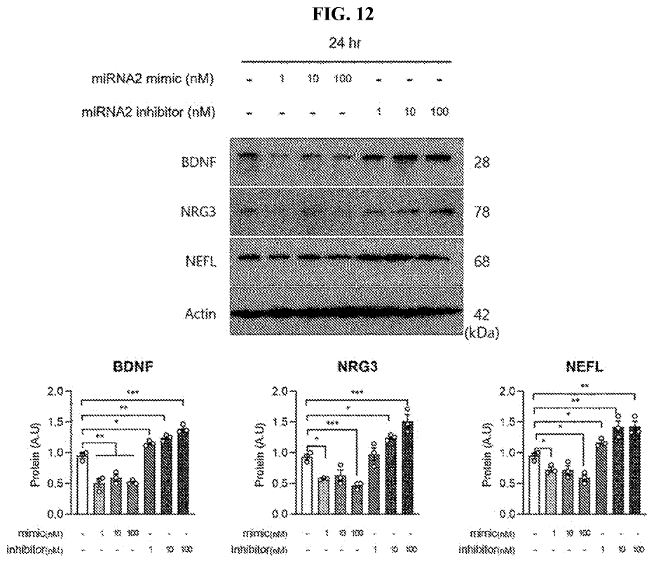

miRNA mimic for the overexpression of novel miRNA and miRNA inhibitor for functional inhibition were separately transfected into human neuroblastoma cells (SH-SY5Y), each at a concentration of 100 nM, for 24 hours. After that, protein separation was carried out, and the expression level was verified using Western blot analysis. As a result, it was found that the overexpression of novel miRNA2 caused more miRNA2 binding to 3′-UTR, resulting in a significant decrease in the expression of candidate target gene proteins BDNF, NRG3 and NEFL, but, with the miRNA inhibitor, a concentration-dependent increase in the expression of the candidate target gene proteins BDNF, NRG3, and NEFL is shown in accordance with the removal of miRNA2 binding to 3′ (FIG. 12).

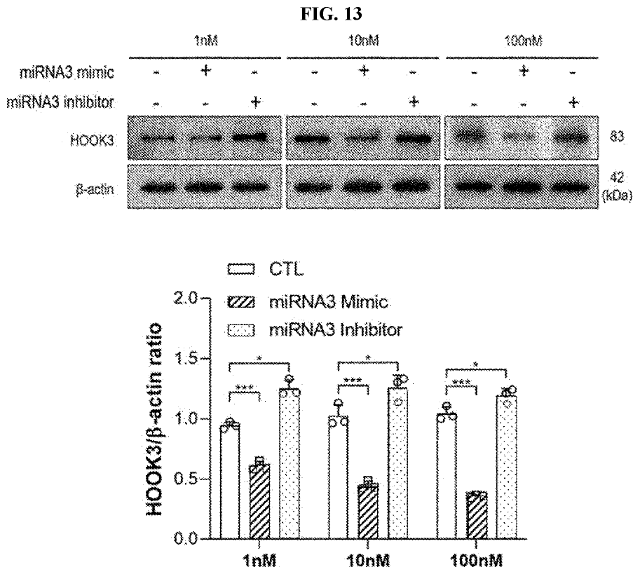

Meanwhile, miRNA3 mimic for overexpression of novel miRNA3 and miRNA3 inhibitor for functional inhibition were separately transfected, each in concentration-dependent manner, into human neuroblastoma SH-SY5Y for 24 hours, and the protein was isolated and subjected to Western blot analysis. As a result, it was found that a treatment with the miRNA3 mimic, which exhibits the overexpression effect of novel miRNA3, showed a significant decrease in expression of the candidate target gene protein HOOK3, and a treatment with miRNA3 inhibitor to remove miRNA3 binding to 3′-UTR of the target gene showed an increase in HOOK3 expression (FIG. 13).

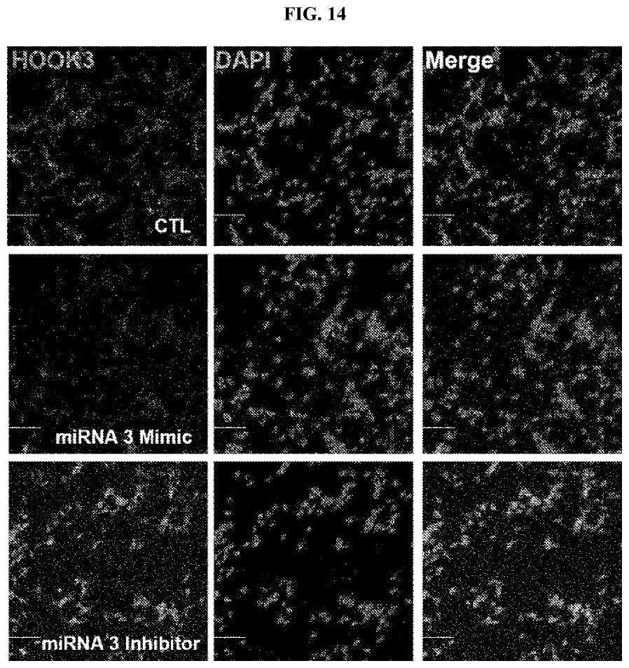

Moreover, after fixing the concentrations of the miRNA mimics and miRNA inhibitors at 1 nM, the expression of target gene HOOK3 protein was compared using immunocytochemistry. The results were found to have the expression patterns that are consistent with Western blot (FIG. 14).

Meanwhile, among the genes regulated by the novel miRNAs, in order to select another target that is highly relevant to Alzheimer type dementia, miRNA4 mimic sequence was screened for each score by using target prediction miRDB software, based on the sequence 5′-GGGGGUGUGGGGUAAAAAAU-3′ (SEQ ID NO: 4), and miRNA5 mimic was also screened for each score based on sequence 5′-GGGGGAGAGAAGGGAAAAG-3′ (SEQ ID NO: 5). The miRNA inhibitor for miRNA mimic of SEQ ID NO: 4 is 5′-AUUUUUUACCCCACACCCCC-3′ (SEQ ID NO: 9), and the miRNA inhibitor for miRNA mimic of SEQ ID NO: 5 is 5′-CUUUUCCCUUCUUCUCUCCCCC-3′ (SEQ ID NO: 10).

After setting the cut-off value of the gene score of the miRNA to 80, target genes related to neurological and Alzheimer type dementia were selected.

The microtubule associated protein 1A (MAP1A) gene was selected as the target gene for miRNA4. The MAP1A protein is a tau-binding protein belonging to the microtubule-related protein family in the brain, and, being involved in the assembly of microtubules, which is an essential step of neurogenesis, plays an important role in regulating homeostasis and synaptic plasticity of neurons.

miRNA4 mimic for overexpression of novel miRNA4 and miRNA4 inhibitor for functional inhibition were separately transfected into human neuroblastoma SH-SY5Y for 24 hours, with each miRNA at different concentrations. After that, the protein was isolated and subjected to Western blot analysis.

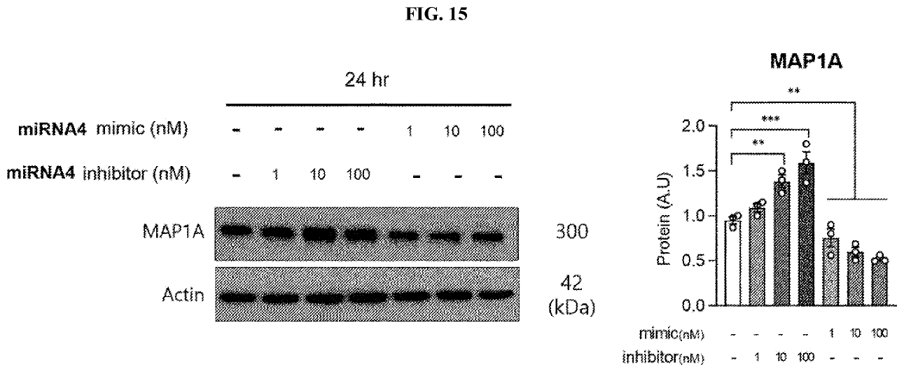

It was found that overexpression of miRNA4 resulted in a significant decrease in the expression of the candidate target gene protein MAP1A due to an increase in miRNA4 binding to 3′-UTR. It was also found that, with the inhibitor miRNA to remove miRNA4 binding to 3′-UTR, an increase in the expression of the target protein MAP1A is shown (FIG. 15).

In addition, SHANK1 (SH3 and multiple ankyrin repeat domains protein 1) was selected as the target gene for miRNA5. SHANK1 protein forms a complex with GKAP/PSD-95 and Homer and plays a structural and functional role in the dendritic spine and synaptic junction as an adapter protein in the postsynaptic density (PSD) that connects the actin-based cytoskeleton to the receptors of the postsynaptic membrane containing NMDA-type and metabolic glutamate receptors.

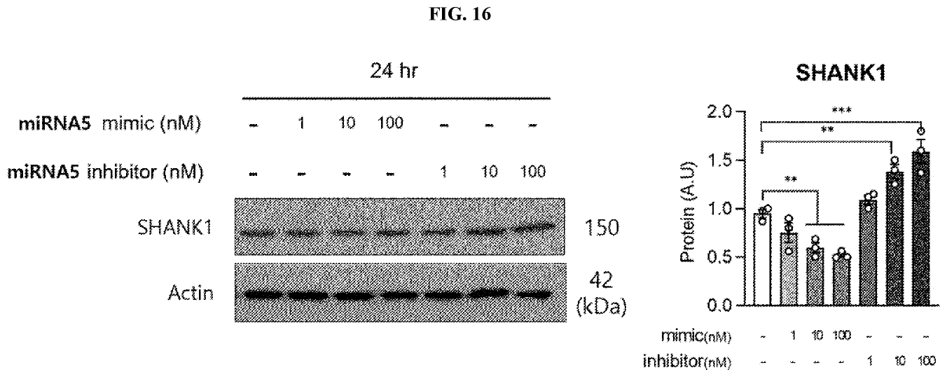

Mimic miRNA5 for overexpression of novel miRNA5 and inhibitor miRNA5 for functional inhibition were separately transfected into human neuroblastoma SH-SY5Y in concentration-dependent manner (1 nM, 10 nM, and 100 nM) for 48 hours, and the protein was isolated and subjected to Western blot analysis. As a result, it was found that, in accordance with the increase in miRNA5 binding to 3′-UTR due as caused by the overexpression of novel miRNA5, a significant decrease in expression of the candidate target gene protein SHANK1 is shown but, by removing the miRNA5 binding to 3′-UTR with inhibitor miRNA5, an increase in the expression of candidate target gene protein SHANK1 is obtained (FIG. 16).

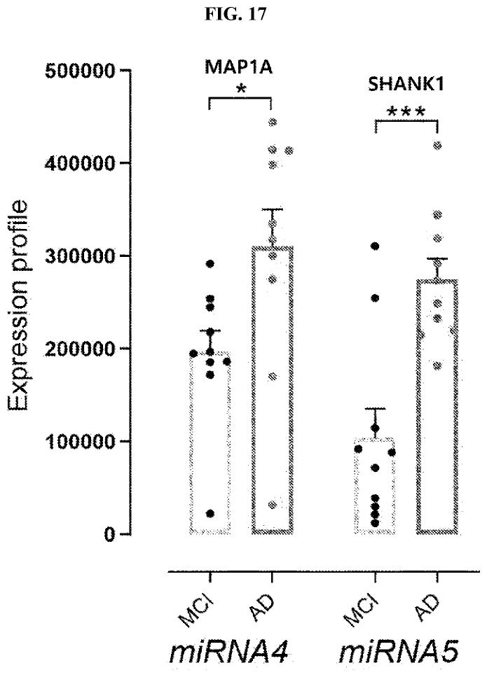

Meanwhile, in order to determine whether or not the expression level of novel miRNAs in the actual patient's blood differs between the different patient groups, target sequencing was carried out for the analysis of the expression level of novel miRNA4 and miRNA5 in the blood plasma of patients who had been classified as MCI or AD. As a result, it was found that there is a significant difference in the expression level of miRNA4 and miRNA5 between the mild cognitive impairment (MCI) group and the Alzheimer type dementia (AD) group (FIG. 17).

### REFERENCE TO AN ELECTRONIC SEQUENCE LISTING

A sequence listing electronically submitted on Feb. 8, 2024 as a XML file named 20240208_S29324GR01_TU_SEQ.XML. created on Feb. 6, 2024, and having a size of 19,359 bytes. is incorporated herein by reference in its entirety.

1-5. (canceled)

6. A biosensor composition for early diagnosis of mild cognitive impairment or Alzheimer type dementia, the biosensor comprising:
an agent capable of detecting any one of miRNAs selected from the group consisting of nucleotide sequences of SEQ ID NOs: 1 to 5 as an effective component.

7. The biosensor composition according to claim 6, wherein the agent contains a nano-fluorescent complex having biotin-streptavidin serving as a linker and a fluorophore of various wavelengths and a quencher bound to the complex.

8. The biosensor composition according to claim 6, wherein the agent is an antisense oligonucleotide, a primer, or a probe complementary to the miRNAs.

9. A kit for early diagnosis of mild cognitive impairment or Alzheimer type dementia, the kit comprising the biosensor composition of claim 6, and the agent comprises an antisense oligonucleotide, a primer, or a probe complementary to the miRNAs, and a nano-fluorescent complex having biotin-streptavidin serving as a linker and a fluorophore of various wavelengths and a quencher bound to the complex.

10. The kit according to claim 9, wherein the kit is used for RT-PCR, real-time PCR, isothermal PCR, Northern blotting, RNA protection assay, or microarray chip.

11. The kit according to claim 9, wherein the kit is used for a medical device for HTS (high-throughput-screening)-based multiplexed POCT (point-of-care-testing).

12. The kit according to claim 11, wherein the medical device for multiplexed POCT includes an incubator, a stirrer, a disposable tip, and a sample injection container.

13. The kit according to claim 11, wherein the medical device for multiplexed POCT is an all-in-one medical device equipped with a micro whole blood separator, a fluorescence detector, and a liquid handler.

14. The kit according to claim 13, wherein the all-in-one medical device equipped with a micro whole blood separator, a fluorescence detector, and a liquid handler includes an algorithm and a detection system for calculating dementia risk index from human blood.

15. (canceled)

16. A method for early diagnosis of mild cognitive impairment or Alzheimer type dementia, the method comprising:
collecting blood from an individual,
separating blood plasma from the blood; and
examining whether any one of miRNAs selected from the group consisting of nucleotide sequences of SEQ ID Nos: 1 to 5 in the blood plasma is expressed or not.

17. A method for early diagnosis of mild cognitive impairment or Alzheimer type dementia, the method comprising:
collecting blood from an individual,
separating blood plasma from the blood; and
examining whether any one of miRNAs selected from the group consisting of nucleotide sequences of SEQ ID Nos: 1 to 5 in the blood plasma is expressed or not by using the biosensor composition of claim 6.

18. The method of claim 17, wherein the agent contains a nano-fluorescent complex having biotin-streptavidin serving as a linker and a fluorophore of various wavelengths and a quencher bound to the complex.

19. The method of claim 17, wherein the agent is an antisense oligonucleotide, a primer, or a probe complementary to the miRNAs.

20. The method of claim 17, wherein the any one of the miRNAs includes SEQ ID NO: 1.

21. The method of claim 17, wherein the any one of the miRNAs includes SEQ ID NO: 2.

22. The method of claim 17, wherein the any one of the miRNAs includes SEQ ID NO: 3.

23. The method of claim 17, wherein the any one of the miRNAs includes SEQ ID NO: 4.

24. The method of claim 17, wherein the any one of the miRNAs includes SEQ ID NO: 5.

25. The method of claim 17, wherein the agent comprises:
an antisense oligonucleotide, a primer, and/or a probe complementary to the miRNAs; and
a nano-fluorescent complex having biotin-streptavidin serving as a linker and a fluorophore of various wavelengths and a quencher bound to the complex.


SEQUENCE LISTING (20 sequences)

SEQ ID NO: 1 - RNA, 20 nt, Homo sapiens
  source 1..20: mol_type = other RNA
caacagagcaagactctgtc

SEQ ID NO: 2 - RNA, 22 nt, Homo sapiens
  source 1..22: mol_type = other RNA
taaagaggaaagaggaaagagg

SEQ ID NO: 3 - RNA, 18 nt, Homo sapiens
  source 1..18: mol_type = other RNA
cgggaggcagcagtgggc

SEQ ID NO: 4 - RNA, 20 nt, Homo sapiens
  source 1..20: mol_type = other RNA
gggggtgtggggtaaaaaat

SEQ ID NO: 5 - RNA, 19 nt, Homo sapiens
  source 1..19: mol_type = other RNA
gggggagagaagggaaaag

SEQ ID NO: 6 - RNA, 20 nt, Homo sapiens
  source 1..20: mol_type = other RNA
gacagagtcttgctctgttg

SEQ ID NO: 7 - RNA, 22 nt, Homo sapiens
  source 1..22: mol_type = other RNA
cctctttcctctttcctcttta

SEQ ID NO: 8 - RNA, 18 nt, Homo sapiens
  source 1..18: mol_type = other RNA
gcccactgctgcctcccg

SEQ ID NO: 9 - RNA, 20 nt, Homo sapiens
  source 1..20: mol_type = other RNA
attttttaccccacaccccc

SEQ ID NO: 10 - RNA, 22 nt, Homo sapiens
  source 1..22: mol_type = other RNA
cttttcccttcttctctccccc

SEQ ID NO: 11 - RNA, 88 nt, synthetic construct
  source 1..88: mol_type = other RNA
taggagaatcgcttgaacccaggaggcggagcttccagtgagctgagatcactgcactccagcctccgcaacagagcaagactctgtc

SEQ ID NO: 12 - RNA, 115 nt, synthetic construct
  source 1..115: mol_type = other RNA
aggttgagataggagaatcgcttgaacccaggaggcggagcttccagtgagctgagatcactgcactccagcctccgcaacagagcaagactctgtctaaaaaaaaaaaaagggc

SEQ ID NO: 13 - RNA, 48 nt, synthetic construct
  source 1..48: mol_type = other RNA
taaagaggaaagaggaaagagttttggaaaatctttttttttttttag

SEQ ID NO: 14 - RNA, 110 nt, synthetic construct
  source 1..110: mol_type = other RNA
aagttcagcccattataaagaggaaagaggaaagaggaaaatctttttttttttttttttgagatggagtcttgctctgtttgatttagagacactagattttttttttt

SEQ ID NO: 15 - RNA, 49 nt, synthetic construct
  source 1..49: mol_type = other RNA
cgggagccgcagcagtgggccaagaccgcaccactgcactagcctgggc

SEQ ID NO: 16 - RNA, 108 nt, synthetic construct
  source 1..108: mol_type = other RNA
ggcaaagaatcgcctgaacccgggaggcagcagtgggccaagaccgcaccactgcactccagcctgggcaacagaatgattccatctcaaaaaaaaaaaaaaaaaaaa

SEQ ID NO: 17 - RNA, 77 nt, synthetic construct
  source 1..77: mol_type = other RNA
gggggtgtggggtaaaaaataacccatctggtacaatgttcactatttgggtaatgggtacactagaagcccaatct

SEQ ID NO: 18 - RNA, 109 nt, synthetic construct
  source 1..109: mol_type = other RNA
aagaggggagggtggaaggggggtgtggggtaaaaaataacccatctggtacaatgttcactatttgggtaatgggtacactagaagcccaatctccaccaatatgcat

SEQ ID NO: 19 - RNA, 66 nt, synthetic construct
  source 1..66: mol_type = other RNA
gggggagagaagggaaaagatccccaagaccccctggggtgggatctgagctcccacctcccttcc

SEQ ID NO: 20 - RNA, 108 nt, synthetic construct
  source 1..108: mol_type = other RNA
agctcgtgggctgggaatgggggagagaagggaaaagatccccaagaccccctggggtgggatctgagctcccacctcccttcccacctactgcactttcccccttcc